(19) United States
(12) Patent Application Publication
(10) Pub. No.: US 2022/0105053 A1
(43) Pub. Date: Apr. 7, 2022

(54) PHARMACEUTICAL COMPOSITION FOR PREVENTION OR TREATMENT OF ACIDOSIS

(71) Applicant: HAIM BIO CO., LTD., Seoul (KR)

(72) Inventors: Yong Bae KIM, Seoul (KR); Sun Young PARK, Seoul (KR); Kyung Seop YUN, Seoul (KR); Min Hee JANG, Gangwon do (KR); Se Young JEONG, Seoul (KR); Eun Jeong KIM, Seoul (KR); Jin Sam LEE, Gyeonggi-do (KR); Hong Yeoul KIM, Seoul (KR)

(73) Assignee: HAIM BIO CO., LTD., Seoul (KR)

(21) Appl. No.: 17/429,575

(22) PCT Filed: Nov. 29, 2019
(86) PCT No.: PCT/KR2019/016720

§ 371 Date: Aug. 9, 2021

(30) Foreign Application Priority Data
Nov. 30, 2018 (KR) 10-2018-0151767
Nov. 30, 2018 (KR) 10-2019-0098083

(51) Int. Cl. A61K 31/11; A61K 31/17; A61K 31/352; A61K 45/06

(52) U.S. Cl. CPC A61K 31/11; A61K 45/06; A61K 31/352; A61K 31/17

(57) ABSTRACT

The present invention relates to a pharmaceutical composition for preventing or treating acidosis. The pharmaceutical composition has a remarkable effect in decreasing the concentration of acid accumulated in an organism and thus is expected to be widely used in the fields of medicine and health.

### TECHNICAL FIELD

The present invention relates to a pharmaceutical composition for preventing or treating acidosis.

### BACKGROUND ART

Acidosis refers to a condition in which the pH is lower than the normal arterial blood pH of 7.4±0.05 (that is, a condition in which the hydrogen ion concentration is high). Acidosis is classified into “respiratory acidosis” and “metabolic acidosis”, and types of metabolic acidosis include diabetic ketoacidosis, lactic acidosis, or poisoning by toxic substances such as salicylic acid, methanol, and ethylene glycol. Among these, lactic acidosis refers to the acidosis that occurs due to disrupted acid-base balance caused in a case where a large amount of lactic acid is produced and accumulated in the body and is defined as a condition in which lactic acid exceeds 45 mg/dL and pH is 7.25 or lower. In a cell, glucose is metabolized in the presence of oxygen to produce energy. However, in a case where glucose is metabolized in the absence of oxygen, lactic acid is produced. Thus, lactic acidosis occurs mostly in a condition of decreased oxygenation in the body, such as hypoxic shock, decreased blood volume, and left ventricle failure; and lactic acid in tissues increases locally even under influence of drugs and toxins (ethanol and methanol) or under increased energy metabolism such as in tumors. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body.

However, for the treatment of lactic acidosis, there is no alternative, which allows the concentration of lactic acid to be regulated with drugs, other than supplying sufficient oxygen. Thus, there is a need to develop a therapeutic agent for the prevention or treatment of lactic acidosis.

Accordingly, the present invention has been made to solve the above-mentioned problems in the prior art and relates to a pharmaceutical composition for preventing or treating acidosis. The pharmaceutical composition of the present invention has a remarkable effect in decreasing the concentration of acid accumulated in an organism and thus is expected to be widely used in the fields of medicine and health.

### Technical Problem

The present invention has been made to solve the above-mentioned problems in the prior art and relates to a pharmaceutical composition for preventing or treating acidosis.

However, the technical problem to be achieved by the present invention is not limited to the above-mentioned problems, and other problems that are not mentioned will be clearly understood by those of ordinary skill in the art from the following description.

### Solution to Problem

Hereinafter, various embodiments described herein are described with reference to the drawing. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, well-known processes and manufacturing techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the invention. Thus, the appearances of the phrase “in one embodiment” or “an embodiment” in various places throughout this specification are not necessarily referring to the same embodiment of the invention. Furthermore, the particular features, configurations, compositions, or characteristics may be combined in any suitable manner in one or more embodiments.

Unless defined otherwise in the specification, all scientific and technical terms as used herein shall have the same meaning as commonly understood by those of ordinary skill in the art to which the present invention belongs.

In an embodiment of the present invention, the term “acidosis” refers to a condition in which the pH is lower than the normal arterial pH of 7.4±0.05 (that is, a condition in which the hydrogen ion concentration is high). Depending on the cause, acidosis is divided into “respiratory acidosis” in which oxygen supply to tissues decreases due to insufficient oxygen absorption in the lungs or decreased blood flow; and “metabolic acidosis” in which the amount of lactic acid increases in the blood or local tissues regardless of the decrease in oxygen level. The causes of respiratory acidosis include shock from bleeding, heart attack, congestive heart failure, pulmonary edema, severe anemia, and the like. Metabolic acidosis is caused by one or more of three mechanisms: acid load, loss of alkali, or impaired renal acid excretion. Examples of a case where acid increases include diabetic ketoacidosis, lactic acidosis, or poisoning by toxic substances such as salicylic acid, methanol, and ethylene glycol.

In an embodiment of the present invention, the term “lactic acidosis” is a type of acidosis and refers to the acidosis that occurs due to disrupted acid-base balance caused in a case where a large amount of lactic acid is produced and accumulated in the body, and is defined as a condition in which lactic acid exceeds 45 mg/dL and pH is 7.45 or lower. In a cell, glucose is metabolized in the presence of oxygen to produce energy. However, in a case where glucose is metabolized in the absence of oxygen, lactic acid is produced. Thus, lactic acidosis occurs mostly in a condition of decreased oxygenation in the body, such as hypoxic shock, decreased blood volume, and left ventricle failure; and lactic acid in tissues increases locally even under influence of drugs and toxins (ethanol and methanol) or under increased energy metabolism such as in tumors. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body. The causes of lactic acidosis include liver disease, kidney disease, diabetes, leukemia, acquired immunodeficiency syndrome (AIDS), glycogen storage disease, drugs and poisons, severe infections (systemic sepsis and meningitis), tumors, several genetic metabolic and mitochondrial diseases affecting normal ATP production and muscular dystrophy, and strenuous exercise.

In an embodiment of the present invention, the term “treatment” refers to a set of acts performed for alleviation and/or amelioration of a target disease. For the purpose of the present invention, the treatment includes an act of eliminating the cause of acidosis in a state in which acidosis has occurred; or an act of improving symptoms of acidosis by decreasing the concentration of acid generated, in a case where it is not possible to eliminate the cause.

In an embodiment of the present invention, the term “pharmaceutical composition” refers to a composition administered for a specific purpose. For the purpose of the present invention, the pharmaceutical composition of the present invention is intended to prevent or treat acidosis, and may comprise a compound involved in such prevention or treatment, and a pharmaceutically acceptable carrier, excipient, or diluent. In addition, the pharmaceutical composition according to the present invention comprises an active ingredient of the present invention in an amount of 0.1% to 50% by weight with respect to the total weight of the composition. Examples of carriers, excipients, and diluents, which may be included in the composition of the present invention, may include but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

In an embodiment of the present invention, the term “administration” means introducing the composition of the present invention to a patient by any suitable way, and the composition of the present invention may be administered via any common route as long as the route allows the composition to reach target tissue. Oral administration, intraperitoneal administration, intravenous administration, intramuscular administration, subcutaneous administration, intradermal administration, intranasal administration, intrapulmonary administration, rectal administration, intracavitary administration, intraperitoneal administration, and intrathecal administration may be mentioned; however, the present invention is not limited thereto. In the present invention, an effective amount may be adjusted depending on various factors including the type of disease, the severity of disease, types, and contents of the active ingredient(s) and other ingredient(s) included in the composition, type of formulation, the patient's age, weight, general health, sex and diet, frequency of administration, route of administration, secretion rate of the composition, duration of treatment, and simultaneously used drug(s). For adults, the therapeutic pharmaceutical composition may be administered to the body in an amount of 50 ml to 500 ml at a time, in which the dose may be 0.1 ng/kg to 10 mg/kg in a case where the active ingredient is a compound, and may be 0.1 ng/kg to 10 mg/kg in a case where the active ingredient is a monoclonal antibody. The administration may be performed 1 to 12 times a day; and in the case of 12 times a day, the administration may be performed once every 2 hours. In addition, the pharmaceutical composition of the present invention may be administered alone or in combination with other therapies known in the art, such as chemotherapy, radiation, and surgery, for the treatment of target cancer stem cells. In addition, the pharmaceutical composition of the present invention may be administered in admixture with other treatments designed to enhance immune responses, for example, adjuvants or cytokines (or nucleic acids encoding cytokines) as is well known in the art. Other standard delivery methods, such as biolistic transfer or ex vivo treatment, may also be used. In the ex vivo treatment, for example, antigen presenting cells (APCs), dendritic cells, peripheral blood mononuclear cells, or bone marrow cells may be obtained from a patient or an appropriate donor and activated ex vivo with the present pharmaceutical composition, and then returned to the patient.

In an embodiment of the present invention, “food composition” is used in various ways for the prevention or amelioration of indications targeted by the present invention. Food compositions comprising the composition of the present invention as an active ingredient may be prepared in the form of various foods, for example, beverages, gums, tea, vitamin complexes, powders, granules, tablets, capsules, confections, rice cakes, bread, and the like. The food composition of the present invention is composed of ingredients that have been obtained by adding improvements to existing food ingredients having little toxicity and side effects and thus can be used without worries in a case of being ingested for a long time for preventive purposes. In a case where the composition of the present invention is included in the food composition, the composition of the present invention may be added in an amount corresponding to a proportion of 0.1% to 100% of the total weight. Here, in a case where the food composition is prepared in the form of a beverage, there is no particular limitation except that the beverage contains the food composition at an indicated proportion, and the beverage may contain various flavoring agents or natural carbohydrates, or the like as additional ingredients similarly to conventional beverages. That is, examples of natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like). In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like. These ingredients may be used individually or in combination. The proportion of these additives is usually selected in a range of 0.1 to 100 parts by weight per 100 parts by weight of the composition of the present invention; however, the present invention is not limited thereto.

In an embodiment of the present invention, there is provided a pharmaceutical composition for preventing or treating acidosis, comprising, as an active ingredient, at least any one selected from the group consisting of citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, retinoic acid, molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

The pharmaceutical composition preferably comprises, as the active ingredient, at least any one selected from the group consisting of molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

The pharmaceutical composition more preferably comprises, as the active ingredient, at least any one selected from the group consisting of benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

In addition, the pharmaceutical composition even more preferably comprises, as the active ingredient, at least any one selected from the group consisting of diethylaminobenzaldehyde (DEAB), disulfiram, and 3-hydroxy-DL-kynurenine.

In the above, the acidosis is preferably metabolic acidosis or respiratory acidosis, with lactic acidosis being more preferred. However, the present invention is not limited thereto.

In another embodiment of the present invention, there is provided a food composition for preventing or ameliorating acidosis, comprising, as an active ingredient, at least any one selected from the group consisting of citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, retinoic acid, molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

The food composition preferably comprises, as the active ingredient, at least any one selected from the group consisting of molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

The food composition more preferably comprises, as the active ingredient, at least any one selected from the group consisting of benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

In addition, the food composition even more preferably comprises, as the active ingredient, at least any one selected from the group consisting of diethylaminobenzaldehyde (DEAB), disulfiram, and 3-hydroxy-DL-kynurenine.

In the above, the acidosis is preferably metabolic acidosis or respiratory acidosis, with lactic acidosis being more preferred. However, the present invention is not limited thereto.

In yet another embodiment of the present invention, there is provided a method for preventing or treating acidosis, comprising administering to a subject in need thereof, a composition that includes, as an active ingredient, at least any one selected from the group consisting of citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, retinoic acid, molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

In the above, the acidosis is preferably metabolic acidosis or respiratory acidosis, with lactic acidosis being more preferred. However, the present invention is not limited thereto.

In still yet another embodiment of the present invention, there is provided a composition for use in a method for preventing or treating acidosis, the composition comprising, as an active ingredient, at least any one selected from the group consisting of CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, retinoic acid, molinate, N-acetyl-N-acetoxy-4-chlorobenzenesulfonamide, phenylglyoxal, benomyl, cis-diamminedichloridoplatinum (CDDP), chlorpropamide, daidzin, pargyline, phospho(enol)pyruvic acid monosodium salt hydrate, kynurenic acid, diethylaminobenzaldehyde (DEAB), disulfiram, and 3-hydroxy-DL-kynurenine.

In the above, the acidosis is preferably metabolic acidosis or respiratory acidosis, with lactic acidosis being more preferred. However, the present invention is not limited thereto.

Hereinafter, the present invention will be described in detail step by step.

### Advantageous Effects of Invention

The present invention relates to a pharmaceutical composition for preventing or treating acidosis. The pharmaceutical composition has a remarkable effect in decreasing the concentration of acid accumulated in an organism and thus is expected to be widely used in the fields of medicine and health.

### BRIEF DESCRIPTION OF DRAWINGS

FIG. 1 illustrates results obtained by evaluating the acidosis-decreasing effect of candidate substances for acidosis treatment, according to an embodiment of the present invention.

### DETAILED DESCRIPTION OF INVENTION

It was checked whether the candidate substances for lactic acidosis treatment, which were screened in the present invention, have a lactic acid concentration-decreasing effect. As a result, it was found that all candidate substances had a lactic acid concentration-decreasing effect. More specifically, in a case of being compared with the positive control (50 μM sodium oxamate) in which the same concentration as the candidate substances is used, it was found that the group consisting of citral, CVT-10216, cyanamide, and retinoic acid had a lactic acid concentration-decreasing effect of about 5%; the group consisting of molinate, N-acetyl-N-acetoxy-4-chlorobenzenesulfonamide, and phenylglyoxane had a lactic acid concentration-decreasing effect of about 10%; the group consisting of benomyl, cis-diamminedichloridoplatinum, chlorpropamide, daidzin, pargyline, phospho(enol)pyruvic acid monosodium salt hydrate, and kynurenic acid had a lactic acid concentration-decreasing effect of about 15%; and the group consisting of diethylaminobenzaldehyde, disulfiram, and 3-hydroxy-DL-kynurenine had a lactic acid concentration-decreasing effect of about 20%.

Hereinafter, the present invention will be described in more detail by way of examples. These examples are only for describing the present invention in more detail, and it will be apparent to those skilled in the art that according to the gist of the present invention, the scope of the present invention is not limited by these examples.

### Example 1: Screening of Candidate Substances for Acidosis Treatment

As a result of screening various candidate substances to develop substances for treating acidosis, the present inventors discovered the substances as shown in Table 1 below.

TABLE 1

| No. | Name of substance |
|---|---|
| 1 | Benomyl |
| 2 | Cis-diamminedichloridoplatinum (CDDP) |
| 3 | Chlorpropamide |
| 4 | Citral |
| 5 | CVT-10216 (3-[[[3-[4-[(methylsulfonyl)amino]phenyl]-4-oxo-4H- |
| | 1-benzopyran-7-yl]oxy]methyl]benzoic acid, or |
| | 3-[[[3-[4-[(methylsulfonyl)amino]phenyl]- |
| | 4-oxo-4H-chromen-7-yl]oxy]methyl]benzoic acid) |
| 6 | Cyanamide |
| 7 | Daidzin |
| 8 | Diethylaminobenzaldehyde (DEAB) |
| 9 | Disulfiram |
| 10 | Molinate |
| 11 | N-acetyl-N-acetoxy-4-chlorobenzenesulfonamide |
| 12 | Pargyline |
| 13 | Phospho(enol)pyruvic acid monosodium salt hydrate |
| 14 | Phenylglyoxal |
| 15 | Retinoic acid |
| 16 | Kynurenic acid |
| 17 | 3-Hydroxy-DL-kynurenine |

### Example 2: Checking of the Acid Concentration-Decreasing Effect of Candidate Substances for Acidosis Treatment

A lactic acid concentration-decreasing effect of the candidate substances as shown in Table 1 was checked.

To this end, first, A549 cancer cells (adenocarcinomic human alveolar basal epithelial cells, Cat #CCL-185) obtained from the American Type Culture Collection (ATCC) were cultured in RPMI 1640 culture medium containing 10% fetal bovine serum and 1% antibiotic-antimycotic agent under an environment at 37° C. and 5% CO₂, and passages were performed every 3 days. Lactic acid is produced in large amounts when glycolysis is activated in hypoxia. Cancer cells, which are densely packed with cells, are very active in energy-consuming activities, and thus are known to produce lactic acid in large amounts.

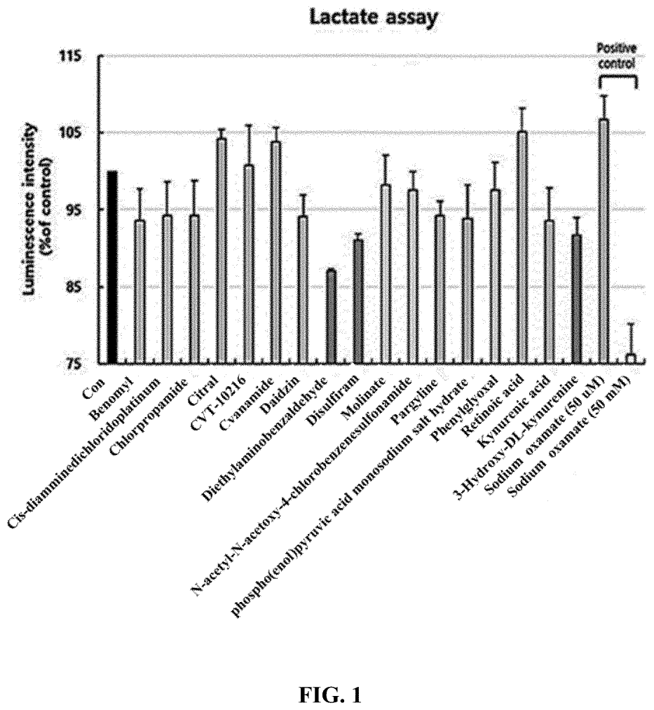

The cells were seeded in 24-well plates at a concentration of 3×10⁵ cells/well, and incubation was performed overnight. Then, each plate was treated with each of the candidate substances (at a concentration of 50 μM) as shown in Table 1 which had been dissolved in dimethyl sulfoxide (DMSO). Incubation was further performed for 24 hours. For the negative control for the candidate substances, no treatment was performed; and for the positive control, treatment with sodium oxamate, which is conventionally known as a therapeutic agent for lactic acidosis, was performed at the same concentration (50 μM) or the 10-fold higher concentration (50 mM). Subsequently, 50 μl of the cell culture solution obtained by being diluted with Dulbecco's Phosphate-Buffered Saline (DPBS), and 50 μl of the lactate assay reaction buffer (Promega, Madison, Wis., USA) were mixed and the mixture was added in a 96-well plate. Reaction was allowed to proceed at room temperature for 1 hour. Then, luminescence was measured with a spectrophotometer (Synergy HTX Multi-Reader, BioTek). At the same time, the number of cells in each sample was measured with a cell viability analysis kit (Cell Counting Kit-8), and calculation was performed so that the lactic acid measurement value in each sample could be compared with that in the negative control for the same number of cells. All experiments were repeated 3 times, and the averages are shown in FIG. 1.

As a result of the experiments, it was found that all candidate substances for lactic acidosis treatment, which were screened in the present invention, had a lactic acid concentration-decreasing effect with only a difference in degree. More specifically, in a case of being compared with the positive control (50 μM sodium oxamate) in which the same concentration as the candidate substances is used, it was found that the group consisting of citral, CVT-10216, cyanamide, and retinoic acid had a lactic acid concentration-decreasing effect of about 5%; the group consisting of molinate, N-acetyl-N-acetoxy-4-chlorobenzenesulfonamide, and phenylglyoxane had a lactic acid concentration-decreasing effect of about 10%; the group consisting of benomyl, cis-diamminedichloridoplatinum, chlorpropamide, daidzin, pargyline, phospho(enol)pyruvic acid monosodium salt hydrate, and kynurenic acid had a lactic acid concentration-decreasing effect of about 15%; and the group consisting of diethylaminobenzaldehyde, disulfiram, and 3-hydroxy-DL-kynurenine had a lactic acid concentration-decreasing effect of about 20%.

Although specific parts of the present invention have been described in detail, it is obvious to those skilled in the art that such a specific description is merely a preferred embodiment, and the scope of the present invention is not limited thereto. Therefore, the substantial scope of the present invention will be defined by the appended claims and equivalents thereof.

### INDUSTRIAL APPLICABILITY

Lactic acidosis occurs mostly in a condition of decreased oxygenation in the body, such as hypoxic shock, decreased blood volume, and left ventricle failure; and lactic acid in tissues increases locally even under influence of drugs and toxins (ethanol and methanol) or under increased energy metabolism such as in tumors. In a case where lactic acidosis continues and acid-base balance is disrupted, symptoms such as muscle weakness, hyperventilation, nausea, vomiting, sweating, or coma may appear and these symptoms may lead to death in severe cases. Therefore, it is important to maintain the acid-base balance by decreasing the concentration of lactic acid that has been over-accumulated in the body.

The pharmaceutical composition of the present invention has a remarkable effect in decreasing the concentration of acid accumulated in an organism and thus is expected to be widely used in the fields of medicine and health.

1-18. (canceled)

19. A method for preventing or treating acidosis in a subject in need thereof, the method comprising administering to the subject a pharmaceutical composition, wherein the pharmaceutical composition comprises at least one active ingredient selected from the group consisting of diethylaminobenzaldehyde (DEAB), disulfiram, 3-hydroxy-DL-kynurenine, benomyl, cis-diamminedichloridoplatinum (CDDP), chlorpropamide, daidzin, pargyline, phospho(enol)pyruvic acid monosodium salt hydrate, kynurenic acid, molinate, N-acetyl-N-acetoxy-4-chlorobenzenesulfonamide, phenylglyoxal, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, and retinoic acid.

20. The method of claim 19, wherein the acidosis is metabolic acidosis or respiratory acidosis.

21. The method of claim 20, wherein the metabolic acidosis is lactic acidosis, diabetic ketoacidosis, or poisoning by a toxic substance.

22. The method of claim 21, wherein the toxic substance is salicylic acid, methanol, or ethylene glycol.

23. The method of claim 21, wherein the metabolic acidosis is lactic acidosis.

24. The method of claim 19, wherein the pharmaceutical composition further comprises a pharmaceutically acceptable carrier.

25. The method of claim 24, wherein the pharmaceutically acceptable carrier is lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, or mineral oil.

26. The method of claim 19, wherein the at least one active ingredient is present in the pharmaceutical composition in an amount of about 0.1% to about 50% by weight.

27. The method of claim 19, wherein the at least one active ingredient is administered to the subject at a dose of about 0.1 ng/kg to about 10 mg/kg of body weight.

28. The method of claim 19, wherein the pharmaceutical composition is administered 1 to 12 times a day.

29. The method of claim 19, wherein the pharmaceutical composition is administered orally, intraperitoneally, intravenously, intramuscularly, subcutaneously, intradermally, intranasally, rectally, intraperitoneally, intrathecally, via intrapulmonary administration, or via intracavitary administration.

30. A method for decreasing acid accumulation in a subject in need thereof, the method comprising administering to the subject a pharmaceutical composition, wherein the pharmaceutical composition comprises at least one active ingredient selected from the group consisting of diethylaminobenzaldehyde (DEAB), disulfiram, 3-hydroxy-DL-kynurenine, benomyl, cis-diamminedichloridoplatinum (CDDP), chlorpropamide, daidzin, pargyline, phospho(enol)pyruvic acid monosodium salt hydrate, kynurenic acid, molinate, N-acetyl-N-acetoxy-4-chlorobenzenesulfonamide, phenylglyoxal, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, and retinoic acid.

31. The method of claim 30, wherein the acid accumulation is lactic acid accumulation.

32. The method of claim 31, wherein the pharmaceutical composition further comprises a pharmaceutically acceptable carrier.

33. The method of claim 32, wherein the pharmaceutically acceptable carrier is lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, or mineral oil.

34. The method of claim 30, wherein the at least one active ingredient is present in the pharmaceutical composition in an amount of about 0.1% to about 50% by weight.

35. The method of claim 30, wherein the at least one active ingredient is administered to the subject at a dose of about 0.1 ng/kg to about 10 mg/kg of body weight.

36. The method of claim 30, wherein the pharmaceutical composition is administered 1 to 12 times a day.

37. The method of claim 30, wherein the pharmaceutical composition is administered orally, intraperitoneally, intravenously, intramuscularly, subcutaneously, intradermally, intranasally, rectally, intraperitoneally, intrathecally, via intrapulmonary administration, or via intracavitary administration.

38. A method for maintaining acid-base balance in a subject in need thereof, the method comprising administering to the subject a pharmaceutical composition, wherein the pharmaceutical composition comprises at least one active ingredient selected from the group consisting of diethylaminobenzaldehyde (DEAB), disulfiram, 3-hydroxy-DL-kynurenine, benomyl, cis-diamminedichloridoplatinum (CDDP), chlorpropamide, daidzin, pargyline, phospho(enol)pyruvic acid monosodium salt hydrate, kynurenic acid, molinate, N-acetyl-N-acetoxy-4-chlorobenzenesulfonamide, phenylglyoxal, citral, CVT-10216 (3-[[[3-[4-[(methylsulfonyl)amino]phenyl]-4-oxo-4H-1-benzopyran-7-yl]oxy]methyl]benzoic acid, or 3-[[[3-[4-[(methylsulfonyl)amino]phenyl]-4-oxo-4H-chromen-7-yl]oxy]methyl]benzoic acid), cyanamide, and retinoic acid.
